(19) United States
(12) Patent Application Publication
(10) Pub. No.: US 2018/0154168 A1
(43) Pub. Date: Jun. 7, 2018

(54) Therapeutic Wavelength Emitter

(71) Applicant: James Clair Nafziger, Wilsonville, OR (US)

(72) Inventor: James Clair Nafziger, Wilsonville, OR (US)

(21) Appl. No.: 15/833,984

(22) Filed: Dec. 6, 2017

Related U.S. Application Data
(60) Provisional application No. 62/430,789, filed on Dec. 6, 2016.

(51) Int. Cl. A61N 5/06; F21V 7/22; F21V 3/04; F21V 19/00

(52) U.S. Cl. CPC A61N 5/06; F21V 7/22; F21V 3/0418; A61N 2005/0659; A61N 2005/0663; A61N 2005/0651; A61N 2005/0654; F21V 19/006

(57) ABSTRACT

A therapeutic wavelength emitter is provided with one or more infrared light bulbs disposed within a case. The case is provided with a transparent lid having a plurality of cuprorivaite particles. The cuprorivaite particles receive light from the infrared light bulbs and emit therapeutic light having a broadband wavelength of 650 to 1200 nanometers

### CROSS-REFERENCE TO RELATED APPLICATION(S)

The present application claims priority to U.S. Provisional Patent Application No. 62/430,789 filed on Dec. 6, 2016, entitled “Therapeutic Wavelength Emitter” the entire disclosure of which is incorporated by reference herein.

### BACKGROUND OF THE INVENTION

### 1. Field of Invention

The present invention relates to the field of therapeutic infrared lamps, and more specifically the present invention relates to a therapeutic infrared lamp with a cuprorivaite element.

### 2. Description of Related Art

Light therapy has been a practiced for thousands of years. Many ancient cultures practiced heliotherapy often to treat various ailments using light from the sun. In modern times, artificial light production methods have become a standard for light therapy. Therapies involving ultraviolet light, laser light, red light, and near infrared lights are used to for pain management, skin treatments, and wound healing.

Specifically, red light or near infrared therapy, sometimes referred to as photobiomodulation, has been found to have many beneficial effects. Photobiomodulation has been found to aid cancer patients in relieving painful side effects of chemotherapy and radiation treatment. In other research, red light therapy has been found to aid the healing of wounds, burns, and other skin conditions.

Heat provided along with the red-light therapy can produce additional benefits. Heat has been known to stimulate protein cells of patients and cause sweating to provide detoxification. While light emitting diodes can emit red light of beneficial wavelengths, they produce little heat. Therefore, they may not be ideal for all light therapy applications.

It has been found that photobiomodulation is maximized at wavelengths between 650-1350 nanometers (nm). Including, but not limited to, maximum vasodilation at 970 nm, deepest penetration into human tissue at 800 nm, and greatest hemoglobin release of oxygen at 905 nm. Unfortunately, most red light therapy bulbs produce wavelengths in the range of 620-700 nm, which lies outside of the range of many beneficial wavelengths. It has been found calcium copper silicate, or cuprorivaite, will emit a photoluminescence with a wavelength range of 800-1200 nm when exposed to red light. This photoluminescence emitted by the cuprorivaite would cover the additional beneficial wavelengths that the red light, alone, would not achieve.

Based on the foregoing, there is a need in the art for a therapeutic wavelength emitter which can produce a wavelength range which covers all beneficial wavelengths. Furthermore, what might be desired is a device which combines emitted light from red bulbs and cuprorivaite photoluminescence to achieve a beneficial wavelength range.

### SUMMARY OF THE INVENTION

In an embodiment of the present invention, a therapeutic wavelength emitter is provided. The therapeutic wavelength emitter is comprised of a case having a transparent lid. One or more light bulb receptacles are disposed within the case and each light bulb receptacle accepts an infrared light bulb. A reflective shield surrounds the light bulb receptacles. In the embodiment, a plurality of cuproriviate particles are adhered to the transparent lid, such that the cuprorivaite particles receive infrared light emitted from the infrared light bulbs and emit light having a broadband wavelength of 650 to 1200 nanometers.

In another embodiment, the cuprorivaite particles are disposed within the transparent lid.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a top plan view of the therapeutic wavelength emitter, according to an embodiment of the present invention;

FIG. 2 is a cross-sectional view taken along line 2-2 in FIG. 1 of the therapeutic wavelength emitter, according to an embodiment of the present invention; and

FIG. 3 is a top plan view of the therapeutic wavelength emitter, according to an embodiment of the present invention.

### DETAILED DESCRIPTION OF PREFERRED EMBODIMENTS

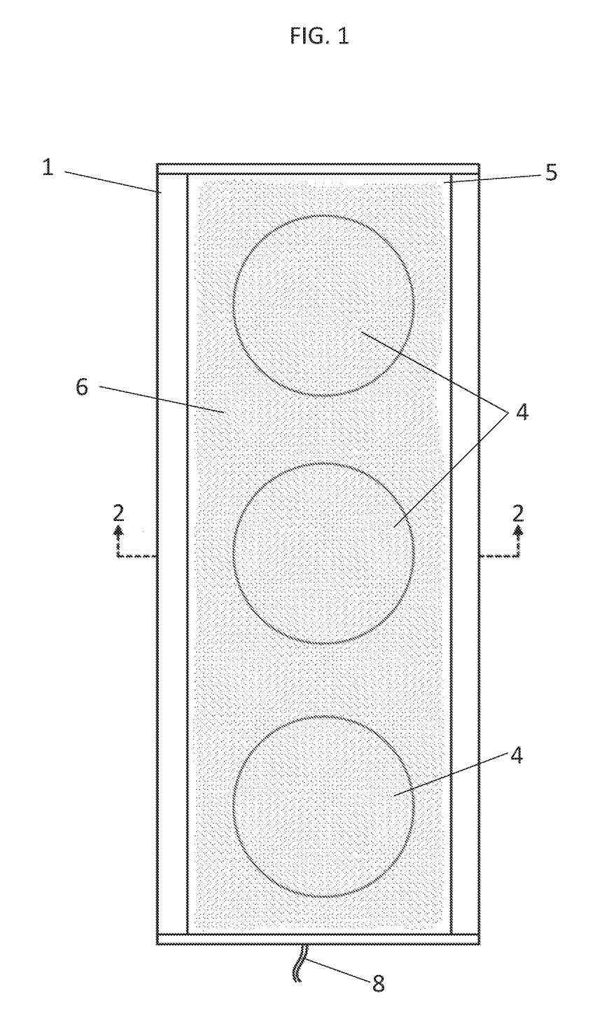

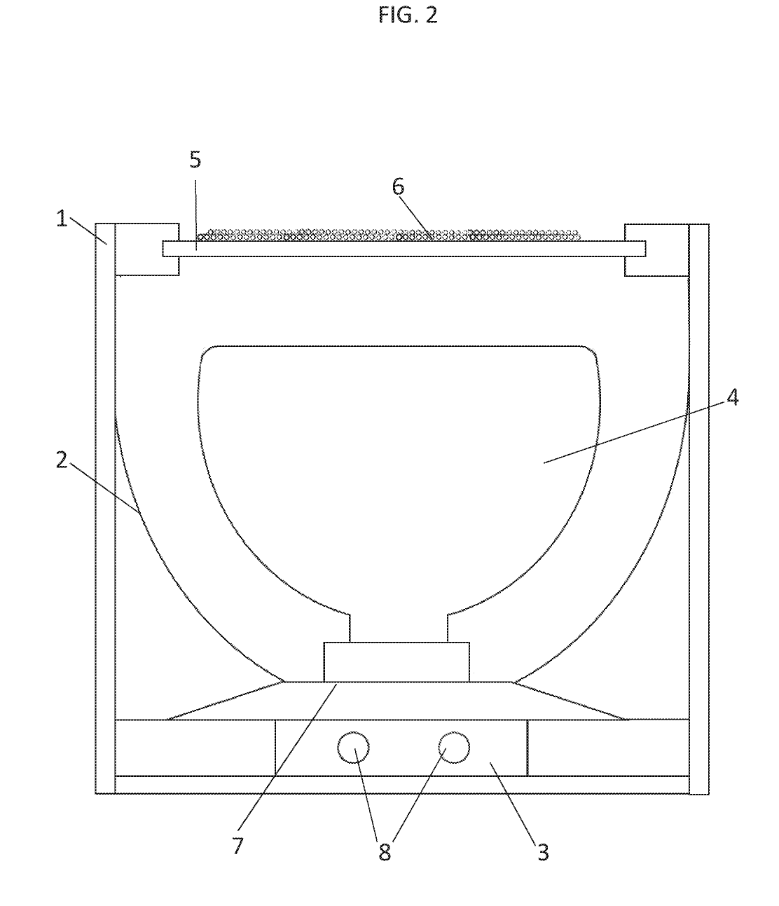

Preferred embodiments of the present invention and their advantages may be understood by referring to FIGS. 1-3, wherein like reference numerals refer to like elements.

In reference to FIGS. 1-3, the therapeutic wavelength emitter is shown comprising of an exterior casing 1. In an example embodiment, the casing 1 is has a general box shape and is comprised of birch plywood. In another embodiment, the casing 1 may be comprised of any type of material and shape deemed appropriate by one skilled in the art. In a preferred embodiment, the casing 1 is fastened together using nails. In another embodiment, the casing 1 may be fastened or adhered by any means deemed appropriate by one skilled in the art. The therapeutic wavelength emitter is further provided with a reflective layer 9 to protect the casing from extensive heat buildup and direct emitted heat. In a preferred embodiment, the reflective layer 9 is comprised of aluminum, which is electrically grounded. In the embodiment, the reflective layer 9 further prevents unwanted electro-magnetic fields from being emitted from the wiring 8 of the device.

In a preferred embodiment, the wiring 8 is provided within an insulated conduit 3. The insulation of the conduit 3 should protect the wiring from heat build-up and prevent unwanted electro-magnetic fields from radiating from the wiring 8. The provided wiring 8 will supply power to a series of heat/red lamp bulbs 4. In an embodiment, the heat/red lamp bulbs are provided onto a base 7. The base 7 will provide one or more electrically active threaded aperture, connected to the wiring 8. The arrangement provides for each heat/red lamp bulbs 4 to be threaded into the base 7 and be supplied with power. In a preferred embodiment, the emitter will be further provided with a toggle switch (not shown) to provide enable a user to turn the toggle the power to the bulbs 4 on and off. In the preferred embodiment, the base will be comprised of a ceramic material to prevent damage due to exposure to heat, but the base may be comprised of any material deemed suitable by one skilled in the art. In a preferred embodiment, the device will accept a 120-volt AC power supply, but it may be configured to accept any suitable power supply.

In a preferred embodiment, the heat/red bulbs 4 are incandescent bulbs capable of producing red light in with wavelengths in the range of 620-750 nm, as known in the art. In another embodiment, the heat/red bulbs 4 may be any bulb type capable of producing red light and heat. In another embodiment, the series of heat/red bulbs may consist of bulbs of different types. For example, two incandescent bulbs may be provided to emit heat and red light, and two red light emitting diodes (LEDs) may be provided, such that all lights emit red light, but only the incandescent bulbs emit heat. In the embodiment, shown in FIG. 1, the therapeutic wavelength emitter is provided with three heat/red bulbs 4. However, any number of bulbs may be used as deemed necessary for the application.

In an example embodiment, the therapeutic wavelength emitter is further provided with a borosilicate glass plate 5 provided above the series of heat/red bulbs 4. The glass plate 5 allows the light from provided bulbs 4 to be emitted onto a user or patient. In an example embodiment, the glass plate 5 is retained by two slots cut within the casing 1. The arrangement is provided such that a user can remove the glass plate 5 by sliding from the slots to replace the bulbs 4.

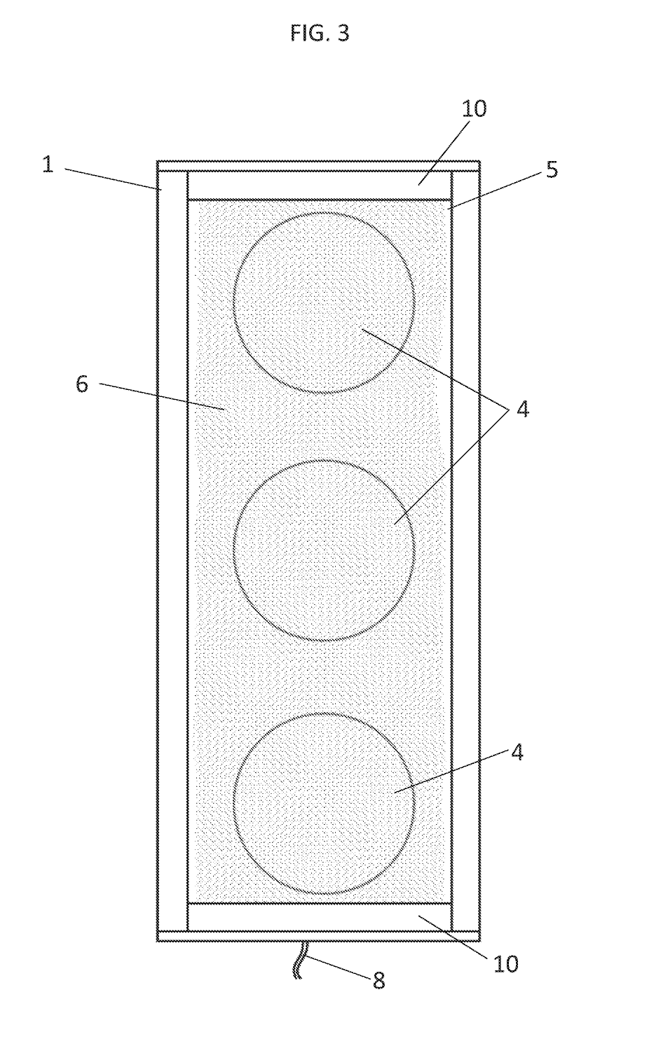

In the embodiment, as shown in FIG. 3, vents 10 are provided between the glass plate 5 and the walls of the casing 1 to allow heat to escape from the casing. In another embodiment,

In an example embodiment, the glass plate 5 is further provided with cuprorivaite particles 6 adhered to the top of the glass plate with a heat resistant adhesive. In another embodiment, the cuprorivaite particles are embedded within the glass plate 5. In an example embodiment, the cuprorivaite particles 6 are dispersed over the glass plate to allow approximately ninety percent of the near infrared (NIR) light to penetrate through the glass plate. The cuprorivaite particles are provided to receive the red light and emit luminescence in a wavelength range of 650-1200 nm.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A therapeutic wavelength emitter comprising:
a case having a transparent lid;
one or more light bulb receptacles disposed within the case;
a reflective shield surrounding the one or more light bulb receptacles; and
a plurality of cuprorivaite particles adhered to the transparent lid,
wherein each of the one or more light bulb receptacles retains an infrared light bulb, and wherein the plurality of cuprorivaite particles receive infrared light emitted from the infrared light bulbs to produce light having a broadband wavelength of 650 to 1200 nanometers.

2. The therapeutic wavelength emitter of claim 1, wherein the one or more light bulb receptacles are comprised of ceramic.

3. The therapeutic wavelength emitter of claim 1, wherein the transparent lid is comprised of glass.

4. The therapeutic wavelength emitter of claim 3, wherein the glass is borosilicate glass.

5. The therapeutic wavelength emitter of claim 1, wherein the case is comprised of birch plywood.

6. The therapeutic wavelength emitter of claim 1, wherein the reflective shield is comprised of aluminum.

7. The therapeutic wavelength emitter of claim 6, wherein the reflective shield is electrically grounded.

8. A therapeutic wavelength emitter comprising:
a case having a transparent lid;
one or more infrared light bulbs disposed within the case, wherein each infrared light bulb is attached to a light base;
a reflective shield surrounding the one or more infrared light bulbs; and
a plurality of cuprorivaite particles disposed within the transparent lid, wherein the plurality of cuprorivaite particles receive infrared light emitted from the one or more infrared light bulbs and emit light having a broadband wavelength of 650 to 1200 nanometers.

9. The therapeutic wavelength emitter of claim 8, wherein the one or more light bulb receptacles are comprised of ceramic.

10. The therapeutic wavelength emitter of claim 8, wherein the transparent lid is comprised of glass.

11. The therapeutic wavelength emitter of claim 10, wherein the glass is borosilicate glass.

12. The therapeutic wavelength emitter of claim 8, wherein the case is comprised of birch plywood.

13. The therapeutic wavelength emitter of claim 8, wherein the reflective shield is comprised of aluminum.

14. The therapeutic wavelength emitter of claim 13, wherein the reflective shield is electrically grounded.

15. A therapeutic wavelength emitter comprising:
a plywood case having a tempered glass lid;
one or more infrared light bulbs disposed within the case, wherein each infrared light bulb is received by a ceramic light base;
an aluminum reflective shield surrounding the one or more infrared light bulbs; and
a plurality of cuprorivaite particles adhered the transparent lid,
wherein the plurality of cuprorivaite particles receive infrared light emitted from the one or more infrared light bulbs and emit light having a broadband wavelength of 650 to 1200 nanometers.

16. The therapeutic wavelength emitter of claim 15, wherein the reflective shield is electrically grounded.
